(19) United States
(12) Patent Application Publication
(10) Pub. No.: US 2026/0152390 A1
(43) Pub. Date: Jun. 4, 2026

(54) METHOD FOR PRODUCING HYDROGEN

(71) Applicant: TOHOKU UNIVERSITY, Sendai-shi (JP)

(72) Inventors: Kouki OKA, Sendai-shi (JP); Takumi Ichimura, Sendai-shi (JP)

(73) Assignee: TOHOKU UNIVERSITY, Sendai-shi (JP)

(21) Appl. No.: 19/400,358

(22) Filed: Nov. 25, 2025

(30) Foreign Application Priority Data
Nov. 29, 2024 (JP) 2024-207873

(51) Int. Cl. C01B 3/00; C01B 3/22; C07C 45/00

(52) U.S. Cl. CPC C01B 3/0015; C01B 3/22; C07C 45/002

(57) ABSTRACT

Provided is a method for producing hydrogen. The method can promote the production of a hydrogen storage compound and the dehydrogenation reaction of the resulting hydrogen storage compound under mild conditions with further reduced cost. The method for producing hydrogen includes hydrogenating a ketone using baker's yeast to obtain a secondary alcohol, and subjecting the secondary alcohol to a dehydrogenation reaction in the presence of a metal complex catalyst to obtain hydrogen.

### CROSS-REFERENCE TO RELATED APPLICATIONS

This application claims priority based on Japanese Patent Application No. 2024-207873 filed Nov. 29, 2024, the disclosure of which is incorporated herein in its entirety by reference.

### TECHNICAL FIELD

The present invention relates to a method for producing hydrogen.

### BACKGROUND ART

Hydrogen gas is expected as next-generation energy that does not emit carbon dioxide when used. To effectively utilize hydrogen, safe transportation of hydrogen is essential. Since hydrogen is in a gas state under ambient conditions, it has a large volume and is easily burned. Thus, it is difficult to transport hydrogen as it is. Thus, currently, in addition to a method of transporting hydrogen gas by compressing hydrogen gas or converting hydrogen gas into a liquid, it is considered to store hydrogen in a hydrogen storage material to transport the hydrogen.

As examples of hydrogen storage materials, metal-organic frameworks (MOF) and hydrogen storage alloys are known. Storage and transportation of hydrogen using these materials require conditions of a low temperature or a high pressure, which costs a lot of money and has a risk of safety.

Meanwhile, organic hydrides are known as hydrogen storage materials. Organic hydrides can safely store hydrogen via chemical bonding under ambient conditions. However, organic hydrides that have been used in previous studies are compounds derived from petroleum such as toluene and fluorenone. In addition, techniques such as reduction through catalytic reactions that have been used in previous studies require a lot of energy for hydrogen storage, and use of expensive precious metal catalysts has been essential.

Further, because the organic hydrides are generally highly volatile liquid, the organic hydrides are inferior in handleability during transportation and storage. Thus, a solid hydrogen storage material has been studied. For example, Patent Document 1 discloses a hydrogen carrier (hydrogen storage material) including a hydrogen storage portion in a main chain and/or a side chain of an organic polymer, in which the hydrogen storage portion generates hydrogen molecules in the presence of a catalyst and becomes an oxidation-reduction active portion, and the oxidation-reduction active portion stores hydrogen trough reduction and contact with a proton source to become the hydrogen storage portion.

### CITATION LIST

### Patent Document

Patent Document 1: WO 2015/005280

### SUMMARY OF INVENTION

### Technical Problem

An object of the present invention is to provide a method for producing hydrogen that can promote the production of a hydrogen storage compound and the dehydrogenation reaction of the resulting hydrogen storage compound under mild conditions with further reduced cost.

### Solution to Problem

The inventors of the present invention have found that a secondary alcohol can be efficiently obtained from a ketone under mild conditions by applying an enzyme reaction using baker's yeast, and extraction of hydrogen (dehydrogenation reaction) from the obtained secondary alcohol can also be promoted with high efficiency under mild conditions. The present invention has been completed by further conducting studies based on these findings.

The object of the present invention have been achieved by the following means.

[1]

A method for producing hydrogen, the method including subjecting a secondary alcohol to a dehydrogenation reaction in the presence of a metal complex catalyst to obtain hydrogen, the secondary alcohol being obtained by hydrogenating a ketone using baker's yeast.

[2]

The method for producing hydrogen according to [1], in which the dehydrogenation reaction is performed at from 150 to 230° C.

[3]

The method for producing hydrogen according to [1] or [2], in which the ketone is at least one of a bicyclomonoketone and a polyhydric ketone.

[4]

The method for producing hydrogen according to any one of [1] to [3], in which the ketone is at least one of a bicyclomonoketone and a chain diketone.

[5]

The method for producing hydrogen according to any one of [1] to [4], in which the ketone is at least one of a bicyclomonoketone and a chain γ-diketone.

[6]

The method for producing hydrogen according to any one of [1] to [4], in which the ketone is at least one of dihydrolevoglucosenone, acetylacetone, and 2,5-hexanedione.

[7]

The method for producing hydrogen according to any one of [1] to [6], in which the metal complex catalyst is an iridium complex catalyst.

[8]

The method for producing hydrogen according to any one of [1] to [7], the method including repeating a cycle a plurality of times, the cycle including hydrogenating, using baker's yeast, the ketone obtained through the dehydrogenation reaction, and subjecting the secondary alcohol obtained through the hydrogenation to the dehydrogenation reaction in the presence of a metal complex catalyst to obtain hydrogen.

In the present invention, a numerical range represented using “(from) . . . to” means a range including numerical values described before and after “to” as a lower limit value and an upper limit value.

### Advantageous Effects of Invention

The method for producing hydrogen of the present invention can promote the production of a hydrogen storage and the dehydrogenation reaction of the resulting hydrogen storage under mild conditions with further reduced cost.

### DESCRIPTION OF EMBODIMENTS

Hereinafter, preferred embodiments of the present invention will be described below, but the present invention is not limited to the following embodiments except for matters defined in the present invention.

### [Method for Producing Hydrogen]

The method for producing hydrogen of the present invention includes subjecting a secondary alcohol obtained by hydrogenating a ketone using baker's yeast to a dehydrogenation reaction in the presence of a metal complex catalyst to obtain hydrogen.

The method for producing hydrogen of the present invention can be an aspect including hydrogenating a ketone using baker's yeast to obtain a secondary alcohol and subjecting the obtained secondary alcohol to the dehydrogenation reaction in the presence of a metal complex catalyst to obtain hydrogen.

The method for producing hydrogen of the present invention is also preferably an aspect in which a cycle is repeated a plurality of times, the cycle including hydrogenating, using baker's yeast, the ketone obtained through the dehydrogenation reaction, and subjecting the secondary alcohol obtained through the hydrogenation to the dehydrogenation reaction in the presence of a metal complex catalyst to obtain hydrogen.

In the method for producing hydrogen of the present invention, the secondary alcohol is obtained by hydrogenating a ketone using an enzyme reaction with baker's yeast, and the hydrogenation through this enzyme reaction is promoted under mild conditions. In the method for producing hydrogen of the present invention, an alcohol having a low molecular weight and a relatively high boiling point can be used as the secondary alcohol. In this case, the secondary alcohol can be handled as a liquid at around ambient temperature. Thus, the secondary alcohol as a hydrogen storage compound can be transported and stored using known transportation means and facilities assuming gasoline or the like. In addition, the secondary alcohol employed in the method for producing hydrogen of the present invention can employ mild conditions as compared with known organic hydrides in a dehydrogenation reaction using a metal complex catalyst.

The method for producing hydrogen of the present invention will be described in more detail.

In the method for producing hydrogen of the present invention, a secondary alcohol obtained by hydrogenating a ketone using baker's yeast is used as a reactive substrate (hydrogen storage compound) for obtaining hydrogen through a dehydrogenation reaction.

The hydrogenation of a ketone using baker's yeast is induced by the action of an enzyme contained in baker's yeast, for example, alcohol dehydrogenase, whereby a reduced coenzyme contained in baker's yeast, for example, nicotinamide adenine dinucleotide (NADH), donates hydrogen to the ketone. As a result, the ketone is reduced to be a secondary alcohol, and NADH is oxidized to be an oxidized coenzyme NAD⁺. More specifically, it is considered that the reaction occurs as in Scheme A described below. The carbonyl group of the ketone is reduced by NADH to become —O⁻, and this —O⁻ is brought into contact with water to extract hydrogen from water, whereby a secondary alcohol is obtained. In Scheme A described below, R₁ and R₂ of (R₁) (R₂)C═O (ketone) are substituents bound at a carbon atom.

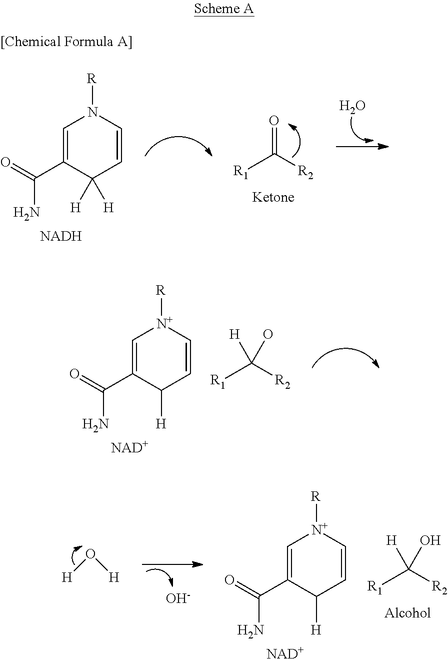

NAD⁺ is reduced through glycolysis of sugars such as glucose to regenerate NADH, and NADH can continuously act on the ketone. Replenishing baker's yeast may allow continuous action of NADH on the ketone.

Nicotinamide adenine dinucleotide phosphate (NADPH) is also thought to act as a coenzyme similarly to NADH.

In a known method for producing an organic hydride, a precious metal catalyst is essential, but in the present invention, the ketone can be hydrogenated without using a precious metal catalyst, which is low cost and can suppress the environmental load.

Hydrogenation of the ketone with baker's yeast is usually performed in water. That is, a part of the hydrogen source of the hydrogenation reaction is water, and hydrogen can be extracted from water present inexhaustibly and stored.

The amount of baker's yeast can be appropriately set according to the type, amount, reaction time, reaction temperature, and the like of the ketone. In general, the reaction proceeds faster as the amount of baker's yeast increases. By increasing the amount of baker's yeast with respect to the amount of ketone moiety of the ketone, the reaction can be performed over a long period of time without regenerating NADH.

For example, baker's yeast can be used in an amount of from 0.5 to 5 g, from 1 to 5 g, or from 1 to 2 g, with respect to 1 mmol of the ketone moiety of the ketone.

The temperature of the hydrogenation reaction of the ketone using baker's yeast is not particularly limited as long as it is a temperature suitable for the enzyme reaction of baker's yeast, and inevitably becomes a mild temperature condition. This hydrogenation reaction can be performed, for example, at from 25 to 40° C., and can also be performed at from 30 to 40° C.

The reaction time of the hydrogenation reaction of the ketone using baker's yeast can be appropriately set according to the type, amount, reaction temperature, and the like of the ketone. The reaction time is not particularly limited. The reaction time of the hydrogenation reaction of the ketone using baker's yeast can be, for example, from 12 to 48 hours or from 12 to 24 hours.

After the hydrogenation reaction, the baker's yeast can be separated through centrifugation or the like.

After the hydrogenation reaction, the secondary alcohol may be separated from water, or it may be directly subjected to a dehydrogenation reaction without being separated. The secondary alcohol can be separated, for example, by evaporating water.

The secondary alcohol obtained as described above can be stored. Thus, the method for producing hydrogen of the present invention may include storing the secondary alcohol.

In addition, the secondary alcohol can be transported. Thus, the method for producing hydrogen of the present invention may include transporting the secondary alcohol.

Since the hydrogen storage compound is a secondary alcohol, handleability is excellent in storage and transportation, and hydrogen can be stored and transported using existing transportation means and facilities.

In the method for producing hydrogen of the present invention, the secondary alcohol obtained as described above is subjected to a dehydrogenation reaction in the presence of a metal complex catalyst to obtain hydrogen. Hydrogen is desorbed and released from the secondary alcohol through the dehydrogenation reaction. At the time, the secondary alcohol is oxidized to be a ketone.

In the dehydrogenation reaction, a solvent may be used or may not be used.

When a solvent is used in the dehydrogenation reaction, the solvent may be water or an organic solvent. Examples of the organic solvent include pentane, hexane, heptane, benzene, toluene, xylene, tetrahydrofuran, diisopropyl ether, dichloromethane, N-methylformamide, 1-butanol, and γ-valerolactone.

From the viewpoint of reducing the environmental load, it is preferable to perform the reaction without a solvent or to use γ-valerolactone, which is a solvent derived from water or cellulose.

The amount of the metal complex catalyst in the dehydrogenation reaction can be appropriately set according to the type, amount, reaction time, reaction temperature, and the like of the secondary alcohol.

For example, the amount of the metal complex catalyst can be, in mole ratio with respect to the secondary alcohol, [metal complex catalyst]/[secondary alcohol]=from 1/1000 to ⅕, from 1/100 to ⅕, from 1/70 to ⅕, or from 1/20 to ⅕.

The temperature of the dehydrogenation reaction is not particularly limited. The dehydrogenation reaction can be performed, for example, at from 100 to 250° C. or at from 150 to 230° C. From the viewpoint of increasing the efficiency of the dehydrogenation reaction, a high-temperature reaction is preferable. The dehydrogenation reaction of methylcyclohexane, a well-known organic hydride, usually requires a high temperature of about from 300 to 400° C.

The reaction time of the dehydrogenation reaction is not particularly limited. The reaction time of the dehydrogenation reaction can be, for example, from 1 to 24 hours, from 1 to 12 hours, or from 1 to 6 hours.

The pH of the reaction liquid for the dehydrogenation reaction is not particularly limited. The pH of this reaction liquid can be, for example, from 1 to 14, from 5 to 14, or from 10 to 14.

In the method for producing hydrogen of the present invention, the hydrogen gas obtained through the dehydrogenation reaction can be recovered by a common method.

After the dehydrogenation reaction, the ketone may be separated from the solvent or may not be separated from the solvent. When the solvent is water, the ketone may be directly subjected to a hydrogenation reaction to be converted into a secondary alcohol without separation.

The ketone generated through the dehydrogenation reaction can be subjected to the hydrogenation reaction again as described above. That is, the ketone can be used as the ketone in the method for producing hydrogen of the present invention.

Thus, in one aspect of the method for producing hydrogen of the present invention, the ketone generated together with hydrogen through the dehydrogenation reaction can be stored as a raw material for the hydrogenation reaction. Thus, the method for producing hydrogen of the present invention may include storing the ketone.

The ketone produced together with hydrogen through the dehydrogenation reaction can be transported. Thus, the method for producing hydrogen of the present invention may include transporting the ketone.

When a solvent is used in the dehydrogenation reaction, the metal complex catalyst after the dehydrogenation reaction can be separated from the solvent through liquid separation, extraction, column chromatography, or the like. The separated metal complex catalyst can be reused as it is. When the metal complex catalyst has been modified or the like, for example, a ligand is added to regenerate the structure of the complex, and the complex can be used again for the dehydrogenation reaction.

In the method for producing hydrogen of the present invention, when a cycle including hydrogenation and dehydrogenation is repeated a plurality of times, a ketone and/or a secondary alcohol, baker's yeast, and a metal complex catalyst can be appropriately replenished in the middle of the cycle.

Hereinafter, the materials used in the method for producing hydrogen of the present invention will be described.

### (Baker's Yeast)

The baker's yeast is not particularly limited as long as it is a yeast normally used for breadmaking. Examples of the baker's yeast include Saccharomyces such as Saccharomyces cerevisiae and Saccharomyces exiguus.

The form of the baker's yeast may be any of fresh yeast, semi-dry yeast, and dry yeast. By allowing baker's yeast in these forms to act on a ketone in the presence of water, a hydrogenation reaction of the ketone occurs through the action of an enzyme.

### (Ketone)

The ketone is not particularly limited as long as it is a compound that generates a secondary alcohol by binding to hydrogen through the action of baker's yeast. The ketone is preferably at least one of a bicyclomonoketone and a polyhydric ketone.

The bicyclomonoketone is a compound having a structure in which two atoms constituting the ring are linked by a bond other than the ring to form a bridge among monoketone having a ring structure (that is, a monoketone having two bridgehead atoms and three bridges connecting them).

In the bicyclomonoketone, the number of ring-constituting atoms including atoms constituting a bridge is preferably from 4 to 10, more preferably from 5 to 9, still more preferably from 6 to 8.

The number of carbon atoms of the bicyclomonoketone is preferably from 3 to 10, more preferably from 4 to 9, still more preferably from 4 to 8.

The bicyclomonoketone may contain a heteroatom (an oxygen atom, a nitrogen atom, or a sulfur atom) as a ring-constituting element, and preferably contains an oxygen atom. Here, the heteroatom contained in the ketone group is not regarded as the ring-constituting element.

The bicyclomonoketone is preferably a compound having from 6 to 8 ring-constituting atoms and containing one or two oxygen atoms as a ring-constituting element.

The molecular weight of the bicyclomonoketone is preferably from 120 to 180, more preferably from 120 to 160, still more preferably from 120 to 140.

The bicyclomonoketone preferably has no aromatic group.

In the bicyclomonoketone, the ring-constituting atom may have a substituent. Examples of the substituent include an alkyl group having from 1 to 10 carbon atoms (preferably from 1 to 8 carbon atoms, more preferably from 1 to 6 carbon atoms, still more preferably from 1 to 4 carbon atoms, still more preferably methyl or ethyl) and a group having an active hydrogen group (—OH, —NH₂, or —SH) (preferably, a hydroxy group).

The bicyclomonoketone is preferably a bicyclomonoketone having from 4 to 10 ring-constituting atoms and containing an oxygen atom as a ring-constituting element.

Specific examples of the bicyclomonoketone include dihydrolevoglucosenone (Cyrene (trade name)), camphor, 3-methylene-2-norbornanone, and 8-oxabicyclo[3,2,1]octane-3-one. Of these, dihydrolevoglucosenone is preferable from the viewpoint of suppressing environmental load because it is a compound derived from cellulose and is biodegradable.

As the polyhydric ketone, a compound having from 1 to 5 ketone groups is preferable, a compound having from 1 to 4 ketone groups is more preferable, a compound having from 1 to 3 ketone groups is still more preferable, and a compound having 2 ketone groups (diketone) is still more preferable.

The polyhydric ketone may be a ketone and at the same time a secondary alcohol. In the present invention, such a compound is also referred to as “ketone”. That is, the polyhydric ketone may have, in addition to the ketone group, a hydroxy group bound to a carbon atom at the center of three consecutive carbon atoms (hydroxy group in an arrangement that the hydroxy group can be oxidized to become a ketone group). That is, in the present invention, the “polyhydric ketone” means that a compound having one ketone group and one hydroxy group in an arrangement that the hydroxy group can become a ketone group as described above is also included in the polyhydric ketone. Thus, ketone K1 in Examples 8 to 11 is also a polyhydric ketone in the present invention.

The polyhydric ketone may be a chain polyhydric ketone or a cyclic polyhydric ketone, and is preferably a chain diketone.

The polyhydric ketone preferably has no aromatic ring in the molecule.

The polyhydric ketone is preferably a ketone having from 5 to 10 carbon atoms, more preferably a ketone having from 4 to 8 carbon atoms, still more preferably a ketone having from 4 to 7 carbon atoms.

The polyhydric ketone preferably has a molecular weight of from 80 to 170, more preferably a molecular weight of from 80 to 150, still more preferably a molecular weight of from 80 to 130, still more preferably a molecular weight of from 80 to 120, and still more preferably a molecular weight of from 100 to 130.

The polyhydric ketone may have a substituent. Examples of the substituent include an alkyl group having from 1 to 10 carbon atoms (preferably from 1 to 8 carbon atoms, more preferably from 1 to 6 carbon atoms, still more preferably from 1 to 4 carbon atoms, still more preferably methyl or ethyl) and a group having an active hydrogen group (—OH, —NH₂, or —SH) (preferably, a hydroxy group).

From the viewpoint of increasing the mass hydrogen density through the hydrogenation reaction, the polyhydric ketone is preferably a compound having a molecular weight of from 80 to 120 and 2 ketone groups (diketone). A diketone, having a high boiling point and low volatility, is preferable from the viewpoint of easy handleability.

Examples of the diketone include an α-diketone, a β-diketone, a γ-diketone, and a 8-diketone. The diketone is preferably a chain diketone. From the viewpoint of conversion, a chain Y-diketone is preferable. The diketone is preferably a chain diketone having from 5 to 10 carbon atoms, more preferably a chain diketone having from 4 to 8 carbon atoms, still more preferably a chain diketone having from 4 to 7 carbon atoms, still more preferably a chain γ-diketone having these numbers of carbon atoms.

Examples of the polyhydric ketone include acetylacetone, 2,5-hexanedione, and 1,4-cyclohexanedione.

The ketone is more preferably at least one of a bicyclomonoketone and a chain diketone, still more preferably at least one of a bicyclomonoketone and a chain γ-diketone, still more preferably at least one of dihydrolevoglucosenone, acetylacetone, and 2,5-hexanedione.

### (Secondary Alcohol)

The secondary alcohol is a compound obtained by hydrogenating the ketone. Thus, the preferred structure of the secondary alcohol is the same as the preferred structure of the ketone except that the carbonyl group in the ketone group of the ketone is converted to a hydroxy group. Thus, a secondary alcohol corresponding to a bicyclomonoketone may be referred to as a bicyclomonoalcohol, and a secondary alcohol corresponding to a polyhydric ketone may be referred to as a polyol. For example, a compound corresponding to a diketone is a diol. In the present invention, the secondary alcohol is a compound having no ketone group.

### (Metal Complex Catalyst)

The metal complex catalyst is not particularly limited as long as it can obtain a ketone and hydrogen from a secondary alcohol through a dehydrogenation reaction in the presence of the metal complex catalyst.

The central metal of the metal complex catalyst is preferably any of iron, copper, vanadium, cobalt, osmium, rhodium, manganese, nickel, iridium, ruthenium, platinum, palladium, and the like, and more preferably iridium.

The ligand of the metal complex catalyst is preferably any of an aqua ligand, a hydroxide ligand, an amine ligand (for example, aniline, toluidine, or anisidine), a diamine ligand (for example, o-phenylenediamine, m-phenylenediamine, or p-phenylenediamine), a pyridine ligand (hydroxypyridine), a bipyridine ligand (for example, 6,6′-dihydroxy-2,2′-bipyridine, 2,2′-bipyridine-6,6′-dionato, or 4,4′-bis(dimethylamino)-2,2′-bipyridine-6,6′-dionato), an acetylacetone ligand (acetylacetonate ligand), a porphyrin ligand, a Schiff base ligand, a phosphine ligand (for example, triphenylphosphine, trimethylphosphine, triethylphosphine, tributylphosphine, tri-tert-butylphosphine, tricyclohexylphosphine, or triethoxyphosphine), a sulfoxide ligand (for example, dimethylsulfoxide), a benzene ligand, a cyclopentadiene ligand (for example, cyclopentadiene or pentamethylcyclopentadiene), and the like, or a combination thereof. Each of the ligands may further have a substituent. Examples of the substituent include an alkyl group having from 1 to 10 carbon atoms and an amino group (for example, an amino group or an alkylamino group having from 1 to 4 carbon atoms).

The metal complex catalyst may be in the form of a salt. For example, it may be a salt with lithium, sodium, potassium, triflate anion (CF₃SO₃⁻), or the like.

The metal complex catalyst is preferably an iridium complex catalyst containing a bipyridine ligand as a ligand thereof, more preferably an iridium complex catalyst having aqua, 6,6′-dihydroxy-2,2′-bipyridine, and pentamethylcyclopentadiene, an iridium complex catalyst having aqua, 2,2′-bipyridine-6,6′-dionato, and pentamethylcyclopentadiene, and an iridium complex catalyst having 4,4′-bis(dimethylamino)-2,2′-bipyridine-6,6′-dionato, and pentamethylcyclopentadiene.

The present invention will be described in more detail with reference to Examples, but the present invention is not to be construed as being limited by these Examples.

### EXAMPLES

### [Material]

Baker's yeast: Nisshin Super Camellia Dry Yeast (available from Nisshin Seifun Welna Inc.)
Dihydrolevoglucosenone (Cyrene (trade name)): available from Sigma-Aldrich Co. LLC.
Acetylacetone: available from FUJIFILM Wako Pure Chemical Corporation
2,5-Hexanedione: available from FUJIFILM Wako Pure Chemical Corporation
1,6-Anhydro-3,4-dideoxy-β-D-threo-hexopyranose: Produced as follows.
Dihydrolevoglucosenone was reduced with LiAlH₄ (available from Tokyo Chemical Industry Co., Ltd.) using dehydrated diethyl ether (available from FUJIFILM Wako Pure Chemical Corporation) as a solvent and purified by column chromatography to produce 1,6-anhydro-3,4-dideoxy-β-D-threo-hexopyranose.
2,4-Pentanediol: available from Tokyo Chemical Industry Co., Ltd.
2,5-Hexanediol: available from Tokyo Chemical Industry Co., Ltd.
Catalyst 1: (aqua (2,2′-bipyridine-6,6′-dionato) (pentamethylcyclopentadienyl) iridium (III)) (available from KANTO CHEMICAL CO., INC.)
Catalyst 2: aqua (6,6′-dihydroxy-2,2′-bipyridine) (pentamethylcyclopentadienyl) iridium (III) bis(triflate) (available from KANTO CHEMICAL CO., INC.)
Toluene: available from FUJIFILM Wako Pure Chemical Corporation
γ-Valerolactone: available from Tokyo Chemical Industry Co., Ltd.

### [Preparation (Hydrogenation) of Hydrogen Storage Compound (Secondary Alcohol)]

The ketone was hydrogenated using baker's yeast (Scheme S1 described below). At this time, 1 g of baker's yeast was used per 1 mmol of the ketone moiety of the ketone. The experimental procedure is as follows, and the conversion was calculated from the following formula by performing 1H NMR measurement. The results are shown in Table 1.

$$Conversion = 100 \times [ molar amount of secondary alcohol ] / ( [ molar amount of ketone ] + [ molar amount of secondary alcohol ] )$$

### Example 1

Baker's yeast in an amount of 1 g was suspended in 10 mL of water and stirred at 30° C. for 1 hour. Thereafter, 1 mmol of dihydrolevoglucosenone as a ketone was added to the suspension, and the mixture was stirred at 30° C. for 24 hours. The ketone was thus hydrogenated to obtain a secondary alcohol (1,6-anhydro-3,4-dideoxy-β-D-threo-hexopyranose) as a hydrogen storage compound.

### Example 2

A ketone was hydrogenated in the same manner as in Example 1 except that the ketone was changed to acetylacetone, and the amount of baker's yeast was changed to 2 g in Example 1, whereby 2,4-pentanediol was obtained as a hydrogen storage compound.

### Example 3

A ketone was hydrogenated in the same manner as in Example 2 except that the ketone was changed to 2,5-hexanedione in Example 2, whereby 2,5-hexanediol was obtained as a hydrogen storage compound.

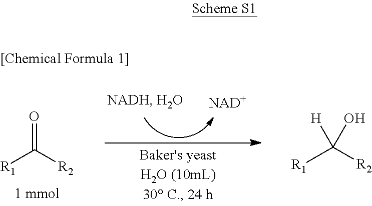

In Scheme S1 described above, R₁ and R₂ of (R₁) (R₂)C═O (ketone) are substituents bound at a carbon atom, and specifically, are substituents corresponding to the ketones shown in Table 1 (the same applies to Scheme S2 to Scheme S4 shown below).

TABLE 1

| | | | Baker′s yeast | Conversion |
|---|---|---|---|---|
| Examples | Ketone | Secondary alcohol | (g) | (%) |
| 1 | 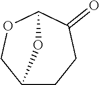 | 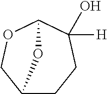 | 1 | 100 |
| 2 | 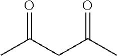 | 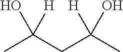 | 2 | 89 |
| 3 | 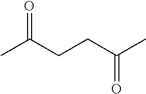 | 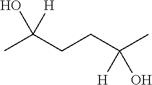 | 2 | 100 |

In Examples 1 and 3, a conversion of 100% was able to be achieved by stirring at 30° C. for 24 hours. Also in Example 2, the conversion was as high as 89%. From the above, it can be seen that both dihydrolevoglucosenone and a polyhydric ketone can be used as raw materials for the hydrogenation reaction using baker's yeast, and a bicyclomonoketone and 2,5-hexanedione (chain γ-diketone) are more preferable from the viewpoint of reaction speed.

### [Production of Hydrogen (Dehydrogenation Reaction): Use of Organic Solvent]

A secondary alcohol was subjected to a dehydrogenation reaction in an organic solvent using the metal complex catalyst (Scheme S2 described below). The experimental procedure is as follows, and the conversion was calculated from the following formula by performing 1H NMR measurement. The results are shown in Table 2.

Conversion=100×[molar amount of ketone]/([molar amount of secondary alcohol]+[molar amount of ketone])

### Example 4

In the air, 1,6-anhydro-3,4-dideoxy-β-D-threo-hexopyranose (1 mmol) was used as a secondary alcohol. The secondary alcohol was added to a flask together with toluene (5 mL) as a solvent and catalyst 1 (molar amount of catalyst 1: molar amount of secondary alcohol=20:100). Thereafter, the reaction solution was heated and stirred at 150° C. for 24 hours under reflux, and the secondary alcohol was subjected to a dehydrogenation reaction to obtain dihydrolevoglucosenone and hydrogen.

### Example 5

A secondary alcohol was subjected to dehydrogenation reaction in the same manner as in Example 4 except that γ-valerolactone was used as a solvent, the reaction temperature was changed to 230° C., and the reaction time was changed to 3 hours in Example 4, whereby dihydrolevoglucosenone and hydrogen were obtained.

### Example 6

A secondary alcohol was subjected to a dehydrogenation reaction in the same manner as in Example 4 except that 2,4-pentanediol was used as the secondary alcohol, the molar amount of the catalyst 1 was changed to 5 with respect to 100 of the molar amount of the secondary alcohol, and the reaction time was changed to 6 hours in Example 4, whereby acetylacetone and hydrogen were obtained.

### Example 7

A secondary alcohol was subjected to a dehydrogenation reaction in the same manner as in Example 6 except that 2,5-hexanediol was used as the secondary alcohol in Example 6, whereby 2,5-hexanedione and hydrogen were obtained.

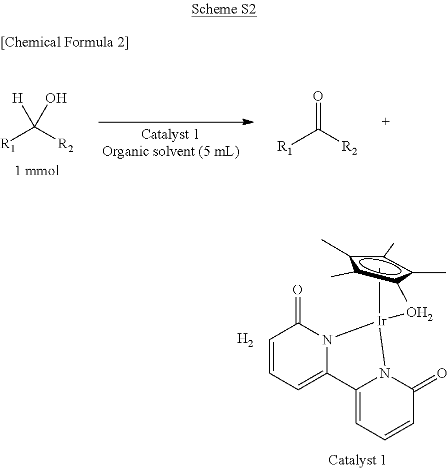

TABLE 2

| | | | Catalyst | | | | |
|---|---|---|---|---|---|---|---|
| | | | (molar | | Temperature | Time | Conversion |
| Examples | Secondary alcohol | Ketone | amount) | Solvent | (° C.) | (h) | (%) |
| 4 | 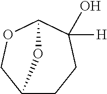 | 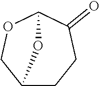 | 20 | Toluene | 150 (Reflux) | 24 | 68 |
| 5 | 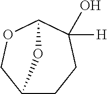 | 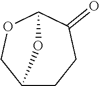 | 20 | γ- Valerolactone | 230 (Reflux) | 3 | 100 |
| 6 | 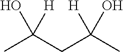 | 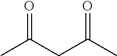 | 5 | Toluene | 150 (Reflux) | 6 | 100 |
| 7 | 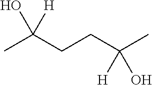 | 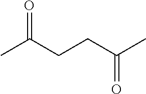 | 5 | Toluene | 150 (Reflux) | 6 | 88 |

*Molar amount: molar amount of catalyst with respect to molar amount 100 of secondary alcohol

The following can be seen from Examples 4 to 7. It can be seen that a ketone and hydrogen are obtained through a dehydrogenation reaction in the presence of an iridium complex catalyst using 1,6-anhydro-3,4-dideoxy-β-D-threo-hexopyranose regardless of the organic solvent species (Examples 4 and 5). In particular, a conversion of 100% was achieved by using γ-valerolactone, which is a solvent derived from cellulose, as an organic solvent and subjecting the secondary alcohol to a high-temperature reaction (Example 5). When toluene was used as the solvent, the conversions of 2,4-pentanediol and 2,5-hexanediol were higher than that of 1,6-anhydro-3,4-dideoxy-β-D-threo-hexopyranose. However, it is considered that, also in Example 7, a conversion of 100% can be achieved also with 2,5-hexanediol by changing the solvent to a solvent having a high boiling point such as γ-valerolactone and subjecting the secondary alcohol to a high-temperature reaction. That is, by subjecting 2,5-hexanediol obtained by hydrogenating 2,5-hexanedione as a chain diketone to dehydrogenation reaction in the presence of a metal complex catalyst to obtain hydrogen, both the hydrogenation reaction and the dehydrogenation reaction can be promoted to a conversion of practically 100%.

### [Production of Hydrogen (Dehydrogenation): Use of Water]

A secondary alcohol was dehydrogenated in water using a metal complex catalyst (Scheme S3 described below). In Scheme S3, OTf⁻ means triflate anion (CF₃SO₃). The experimental procedure is as follows. The results are shown in Table 3.

### Example 8

In the air, 2,4-pentanediol (1 mmol) was used as a secondary alcohol. The secondary alcohol was added to a flask together with water (5 mL) and catalyst 1 (molar amount of catalyst 1: molar amount of secondary alcohol=5:100). Thereafter, the reaction solution was heated and stirred at 150° C. for 24 hours under reflux, and the secondary alcohol was subjected to a dehydrogenation reaction to obtain ketone K1 and ketone K2 shown in Table 3 and hydrogen.

### Example 9

Ketone K1, ketone K2, and hydrogen were obtained by subjecting the secondary alcohol to a dehydrogenation reaction in the same manner as in Example 8 except that catalyst 2 was used instead of catalyst 1 in Example 8.

### Example 10

A secondary alcohol was subjected to a dehydrogenation reaction in the same manner as in Example 8 except that 2,5-hexanediol was used as the secondary alcohol in Example 8, whereby ketone K1, ketone K2, and hydrogen were obtained.

### Example 11

Ketone K1 and ketone K2 were obtained by subjecting the secondary alcohol to a dehydrogenation reaction in the same manner as in Example 10 except that catalyst 2 was used instead of catalyst 1 in Example 10.

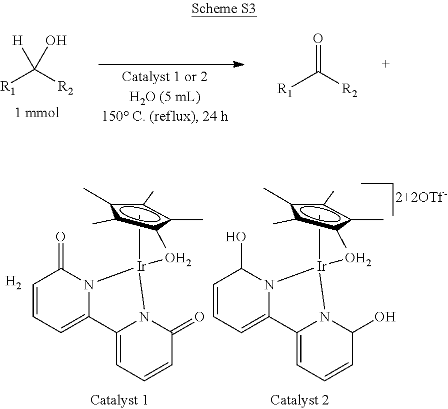

TABLE 3

| | | | | Catalyst | Conversion (%) | |
|---|---|---|---|---|---|---|
| | | | | (molar | Ketone | Ketone |
| Examples | Secondary alcohol | Ketone K1 | Ketone K2 | amount) | K1 | K2 |
| 8 | 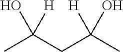 | 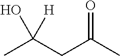 | 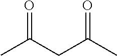 | Catalyst 1 (5) | 75 | 15 |
| 9 | 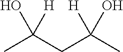 | 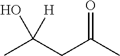 | 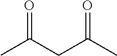 | Catalyst 2 (5) | 21 | 0 |
| 10 | 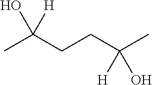 | 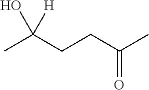 | 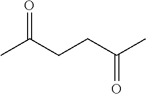 | Catalyst 1 (5) | 27 | 73 |
| 11 | 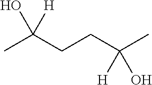 | 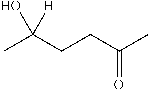 | 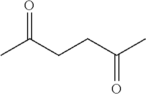 | Catalyst 2 (5) | 39 | 12 |

*Molar amount: molar amount of catalyst with respect to molar amount 100 of secondary alcohol

From Examples 8 to 11, it can be seen that the dehydrogenation reaction proceeds in the presence of an iridium complex catalyst even when water is used as the solvent. Further, it can also be seen that catalyst 1 is more suitable than catalyst 2 to increase the production efficiency of ketone K2 in which all the hydroxy groups are oxidized to be carbonyl groups. It can also be seen that the reaction using 2,5-hexanediol (reaction for obtaining 2,5-hexanedione which is a chain γ-diketone) has a higher conversion than the reaction using 2,4-pentanediol.

It has been confirmed that in the case of 1,6-anhydro-3,4-dideoxy-β-D-threo-hexopyranose, the catalytic reaction does not sufficiently proceed in water when catalysts 1 and 2 are used.

### [Production of Hydrogen (Dehydrogenation): No Solvent is Used]

A secondary alcohol was dehydrogenated under solvent-free conditions using a metal complex catalyst (Scheme S4 described below). The experimental procedure is as follows. The results are shown in Table 4.

### Example 12

In the air, 1,6-anhydro-3,4-dideoxy-β-D-threo-hexopyranose (10 mmol) as a secondary alcohol and catalyst 1 (molar amount of catalyst 1: molar amount of secondary alcohol=5:100) were added to a flask. Thereafter, the reaction solution was heated and stirred at 200° C. for 24 hours, and the secondary alcohol was subjected to a dehydrogenation reaction to obtain dihydrolevoglucosenone and hydrogen.

### Example 13

2,5-Hexanedione and hydrogen were obtained in the same manner as in Example 12 except that 30 mmol of 2,5-hexanediol was used as the secondary alcohol, the molar amount of catalyst 1 was changed to 1.5 with respect to the molar amount of 100 of the secondary alcohol, and the mixture was heated and stirred at 220° C. under reflux in Example 12.

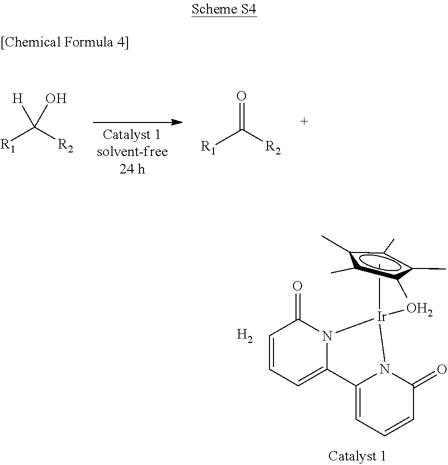

TABLE 4

| | | | Catalyst | | |
|---|---|---|---|---|---|
| | | | (molar | Temperature | Conversion |
| Examples | Secondary alcohol | Ketone | amount) | (° C.) | (%) |
| 12 | 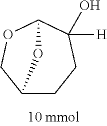 | 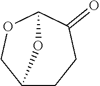 | 5 | 200 | 44 |
| 13 | 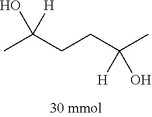 | 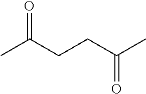 | 1.5 | 220 (Reflux) | 88 |

*Molar amount: molar amount of catalyst with respect to molar amount 100 of secondary alcohol

From Examples 12 and 13, it can be seen that both 1,6-anhydro-3,4-dideoxy-β-D-threo-hexopyranose and 2,5-hexanediol can be dehydrogenated through a dehydrogenation reaction in the presence of an iridium complex catalyst without using a solvent.

In addition, it can be seen from Examples 1 to 13 that hydrogen can be obtained by hydrogenating a ketone using baker's yeast and subjecting the obtained secondary alcohol to a dehydrogenation reaction in the presence of a metal complex catalyst.

1. A method for producing hydrogen, the method comprising subjecting a secondary alcohol to a dehydrogenation reaction in the presence of a metal complex catalyst to obtain hydrogen, the secondary alcohol being obtained by hydrogenating a ketone using baker's yeast.

2. The method for producing hydrogen according to claim 1, wherein the dehydrogenation reaction is performed at from 150 to 230° C.

3. The method for producing hydrogen according to claim 1, wherein the ketone is at least one of a bicyclomonoketone and a polyhydric ketone.

4. The method for producing hydrogen according to claim 1, wherein the ketone is at least one of a bicyclomonoketone and a chain diketone.

5. The method for producing hydrogen according to claim 1, wherein the ketone is at least one of a bicyclomonoketone and a chain γ-diketone.

6. The method for producing hydrogen according to claim 1, wherein the ketone is at least one of dihydrolevoglucosenone, acetylacetone, and 2,5-hexanedione.

7. The method for producing hydrogen according to claim 1, wherein the metal complex catalyst is an iridium complex catalyst.

8. The method for producing hydrogen according to claim 1, the method comprising repeating a cycle a plurality of times, the cycle including hydrogenating, using baker's yeast, the ketone obtained through the dehydrogenation reaction, and subjecting the secondary alcohol obtained through the hydrogenation to the dehydrogenation reaction in the presence of a metal complex catalyst to obtain hydrogen.
